(19) United States
(12) Patent Application Publication
(10) Pub. No.: US 2024/0177870 A1
(43) Pub. Date: May 30, 2024

(54) SYSTEM AND METHOD OF PREDICTING BIOLOGICAL ACTIVITIES OF CHEMICAL COMPOUND PRESENT IN A NEW DRUG

(71) Applicant: Innoplexus AG, Eschborn (DE)

(72) Inventors: Om Sharma, Pimpri-Chinchwad (IN); Ansh Gupta, Pimpri-Chinchwad (IN); Hari Kapa, Vijayawada (IN); Sandhya V, Bengaluru (IN)

(73) Assignee: Innoplexus AG, Eschborn (DE)

(21) Appl. No.: 18/060,302

(22) Filed: Nov. 30, 2022

(51) Int. Cl. G16H 70/40

(52) U.S. Cl. CPC G16H 70/40

(57) ABSTRACT

A system for predicting biological activities or properties of one or more chemical compounds in a new drug. The system includes a processor configured to receive a plurality of input datasets including a plurality of simplified molecular-input line-entry system (SMILES) notations and execute a pre-trained Natural Language Processing (NLP) model to transform the plurality of SMILES notations into a plurality of non-sparse matrices. The processor is configured to train a deep learning model using the plurality of non-sparse matrices to obtain a trained deep learning model, where the trained deep learning model is used to predict one or more biological activities of an untested chemical compound in the new drug when the chemical compound in the drug is subjected to at least one chemical modification to form the new drug. The system efficiently and reliably predicts the biological activities or properties of the untested chemical compound in the new drug.

### TECHNICAL FIELD

The aspects of the disclosed embodiments relate generally to the field of drug design technology; and more specifically, to systems and methods of predicting biological activities or properties of one or more chemical compounds in a new drug.

### BACKGROUND

Generally, planning and execution of a clinical trial process of a drug requires a lot of time, cost, intellectual capital and have a high probability of failure. The drug efficacy and safety issues are considered as major concerns to determine whether to continue or not with the clinical trial process. This is estimated that close to 50% of drug candidates fail because of unacceptable efficacy and approximately 40% drug candidates fail due to toxicity issues. For a successful execution of the clinical trial process, optimization of both the drug efficacy and safety parameters is required at each and every stage of the clinical trial process. Typically, around 33% of human proteins is intrinsically disordered and lacks well established three-dimensional (3D) structures. Therefore, novel drugs cannot be elucidated by a conventional structure-based drug design approach.

Currently, certain attempts have been made to predict biological activities of certain chemical compounds present in a drug, for example, by a structural similarity. The structural similarity between known chemical compounds (or chemical composition) is determined to predict the biological activities of any similar chemical compounds. The conventional methods have only limited utility in practice as such conventional methods determine the structural similarity either by drawing a comparison between sequences of the known chemical compounds and the one or more chemical compounds of the drug using tanimoto coefficients or by measuring root mean square deviation (RMSD) using a 3D structural similarity. However, aforementioned methods inefficiently discriminate the changes in biological activities of the one or more chemical compounds when the known chemical compounds are subjected to a change in any functional group (e.g., change in a bond connecting an atomic pair), and the like. In such cases, conventional systems and methods become unreliable and of limited practical use. Conventionally, structural activity relationship (SAR) models are developed based on different type of descriptors and fingerprints. For example, a one-dimensional (1D) descriptor, a two-dimensional (2D) descriptor, and a three-dimensional (3D) descriptor, is developed to quantify the properties of chemical compounds on the basis of level of molecular representation required for computing a descriptor. For example, the 1D descriptor represents information calculated from molecular formula of a molecule, which includes count and type of atoms in the molecule and the molecular weight. The 2D descriptor represents molecular information regarding the size, shape and electronic distribution in the molecule. Similarly, the 3D descriptor describes the properties related to the 3D conformation of the molecule, such as intramolecular hydrogen bonding. Additionally, fingerprints are an abstract representation of certain structural features of the molecule, including count of a particular atom type (e.g., halogen atom, nitrogen atom, and the like) or detecting the presence of a particular ring system (e.g., phenyl, pyridyl, naphthyl, and the like). The different descriptors and fingerprints are based on the connectivity of atoms and presence or absence of chemical groups resulting in generation of a sparse matrix with a large number of zeros. Thus, it becomes a technical challenge to deal with colossal diversity of data (i.e., determining biological activities or various properties of chemical compounds present in the drug) using a conventional sparse matrix. Thus, there exists a technical problem of how to efficiently and reliably predict any change in biological activities of the one or more chemical compounds present in the drug and thereby saving time, cost and efforts involved in the design and discovery of a new drug.

Therefore, in light of the foregoing discussion, there exists a need to overcome the aforementioned drawbacks associated with the conventional methods of determining the biological activities of the one or more chemical compounds present in the drug.

### SUMMARY

The aspects of the disclosed embodiments are directed to systems and methods of predicting biological activities or properties of one or more chemical compounds in a new drug. An aim of the disclosed embodiments is to provide improved methods and systems of predicting biological activities or properties of one or more chemical compounds in a new drug.

One or more advantages of the disclosed embodiments are achieved by the solutions provided in the enclosed independent claims. Advantageous implementations of the present disclosure are further defined in the dependent claims.

In an aspect, the aspects of the disclosed embodiments provide a system for predicting biological activities or properties of one or more chemical compounds in a new drug. The system comprises a processor configured to receive a plurality of input datasets, wherein the plurality of input datasets comprises a plurality of simplified molecular-input line-entry system (SMILES) notations associated with a plurality of drugs, wherein each of the plurality of SMILES notations is an indicative of a chemical structure of a chemical compound present in a drug. The processor is further configured to execute a pre-trained Natural Language Processing (NLP) model to transform the plurality of SMILES notations into a plurality of non-sparse matrices, wherein each of the plurality of non-sparse matrices is indicative of one or more biological activities of the chemical compound present in the drug. The processor is further configured to train a deep learning model using the plurality of non-sparse matrices to obtain a trained deep learning model, wherein the trained deep learning model is used to predict one or more biological activities of an untested chemical compound in the new drug when the chemical compound in the drug is subjected to at least one chemical modification to form the new drug.

The disclosed system efficiently and reliably predicts the biological activities or properties of the chemical compound present in the drug by virtue of using the pre-trained NLP model and training of the deep learning model. The prediction of the one or more biological activities and one or more physiochemical properties (e.g., melting point, boiling point, solubility, and the like) of the chemical compound in the drug with improved accuracy not only reduces the cost of the drug discovery but also has a direct positive impact on an individual's health. For example, if the bioactivity of a potential drug is estimated in advance, then it can be decided whether the drug can be selected for further studies or not, thereby saving cost, time and efforts. Since the one or more biological activities or properties of the chemical compound are diverse in comparison to the structural properties, drawing a reliable relation between the one or more biological activities and the structural properties of the chemical compound is a tedious task. On the contrary, the disclosed system with the pre-trained NLP model and the deep learning model, manifests an improved prediction accuracy and hence, draws the reliable relation between the one or more biological activities and the structural properties of the chemical compound, further makes the process of drug development more reliable. Conventionally, a drug is biased for use in a particular sale line only and the drug is found as a failure, when used in a different sale line. In contrast to the conventional drug, the disclosed system removes the biasness of the drug and on experimentation, the drug is found to be applicable for use in different sale lines. Moreover, the disclosed system can efficiently and reliably predict the one or more biological activities or properties of the untested chemical compound when the chemical compound already present in the drug is subjected to a change (e.g., 1% change in one of the structural features) to form the new drug by virtue of using the pre-trained NLP model and training of the deep learning model. In other words, conventionally, structural similarity between chemical compounds may be used to predict certain biological activities of chemical compositions or compounds present in drug. Suppose a new drug exhibits 2% change in chemical composition then, by use of structural similarity it was difficult and technically challenging to predict any change in biological properties of one or more chemical compounds present in a new or novel drug. In the present disclosure, instead of structural similarity, a non-sparse matrix is generated by the disclosed system using a transformer model (i.e., the pre-trained NLP model), which is then used to train the deep learning model to predict biological activity and/or properties of previously untested chemical compound of drug prior to experimental synthesis, thereby saving time, cost and efforts involved in traditional methods of drug design and discovery.

In another aspect, the aspects of the disclosed embodiments provide a method of predicting biological activities or properties of one or more chemical compounds in a new drug. In one embodiment, the method comprises receiving, by a processor, a plurality of input datasets, wherein the plurality of input datasets comprises a plurality of simplified molecular-input line-entry system (SMILES) notations associated with a plurality of drugs, wherein each of the plurality of SMILES notations is an indicative of a chemical structure of a chemical compound present in a drug of the plurality of drugs. The method further comprises executing, by the processor, a pre-trained Natural Language Processing (NLP) model to transform the plurality of SMILES notations into a plurality of non-sparse matrices, wherein each of the plurality of non-sparse matrices is indicative of one or more biological activities of the chemical compound present in the drug. The method further comprises training, by the processor, a deep learning model using the plurality of non-sparse matrices to obtain a trained deep learning model, wherein the trained deep learning model is used to predict one or more biological activities of an untested chemical compound present in the new drug when the chemical compound is subjected to at least one chemical modification to form the new drug.

The method achieves all the advantages and technical effects of the disclosed system of the present disclosure.

In another aspect, the aspects of the disclosed embodiments provide a method of predicting biological activities or properties of one or more chemical compounds in a new drug. The method comprises receiving, by a processor, a simplified molecular-input line-entry system (SMILES) notation, wherein the SMILES notation is an indicative of a chemical structure of an untested chemical compound present in the new drug. The method further comprises executing, by the processor, a pre-trained Natural Language Processing (NLP) model to transform the SMILES notation into a non-sparse matrix and executing, by the processor, a trained deep learning model, by passing the non-sparse matrix to the trained deep learning model to predict one or more biological activities and one or more physiochemical properties of the untested chemical compound in the new drug.

The method can reliably predict the biological activities or properties of the untested chemical compound when the chemical compound already present in the drug is subjected to a change (e.g., 1% change in one of the structural features) to form the new drug by virtue of using the pre-trained NLP model and the trained deep learning model.

It is to be appreciated that all the aforementioned implementation forms can be combined. It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1A is a block diagram of a system of predicting biological activities or properties of one or more chemical compounds in a new drug, in accordance with an embodiment of the present disclosure;

FIG. 1B is a block diagram of a system of predicting biological activities or properties of one or more chemical compounds in a new drug, in accordance with another embodiment of the present disclosure;

FIGS. 2A to 2D collectively is a flow chart of a method of predicting biological activities or properties of one or more chemical compounds in a new drug, in accordance with an embodiment of the present disclosure;

FIG. 2E is a flowchart of a method of predicting biological activities or properties of one or more chemical compounds in a new drug, in accordance with an embodiment of the present disclosure;

FIGS. 3A-3B collectively illustrate how to predict bioactivity of one or more chemical compounds present in a drug, in accordance with an embodiment of the present disclosure;

FIG. 4A is a graphical representation that illustrates comparison of time complexity between a transformer model and a conventional PaDEL-descriptor model, in accordance with an embodiment of the present disclosure; and

FIG. 4B is a graphical representation that illustrates comparison of space complexity between a transformer model and a conventional PaDEL-descriptor model, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

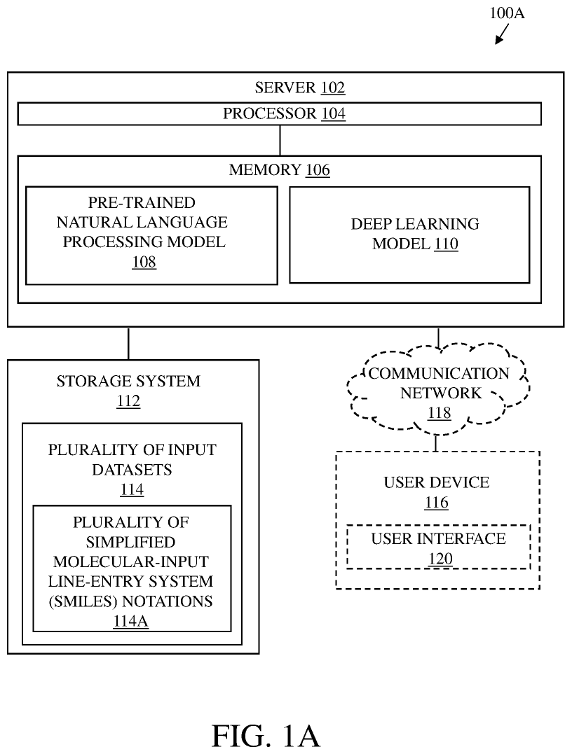

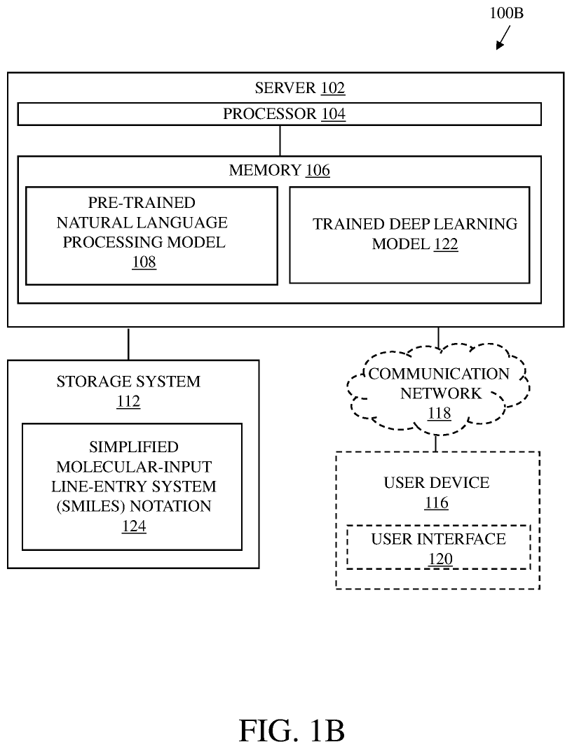

FIG. 1A is a block diagram of a system of predicting biological activities or properties of one or more chemical compounds in a new drug, in accordance with an embodiment of the present disclosure. With reference to FIG. 1A, there is shown a block diagram of a system 100A that includes a server 102. The server 102 includes a processor 104 communicably coupled to a memory 106. The memory 106 includes a pre-trained natural language processing (NLP) model 108 and a deep learning model 110. The memory 106 is further connected to a storage system 112 configured to store a plurality of input datasets 114. The plurality of input datasets 114 comprises a plurality of simplified molecular-input line-entry system (SMILES) notations 114A. Optionally, the server 102 is connected to a user device 116 through a communication network 118. The user device 116 comprises a user interface 120.

The system 100A is used for predicting biological activities or properties of one or more chemical compounds present in a new drug. The system 100A comprises the server 102 that is configured to execute the prediction of predicting biological activities or properties of one or more chemical compounds present in the new drug. Examples of implementation of the server 102 may include, but are not limited to, a storage server, a cloud-based server, a web server, an application server, or a combination thereof. Conventionally, structural similarity between chemical compounds is used to predict one or more biological activities of chemical compositions present in drug. Suppose a novel drug exhibits 2% change in chemical composition then, by use of structural similarity it was difficult and technically challenging to predict any change in biological properties of one or more chemical compounds present in a new or novel drug. In the present disclosure, instead of structural similarity, a non-sparse matrix is generated by the system 100A using a transformer model (i.e., the pre-trained NLP model 108), which is then used to train the deep learning model 110 to predict biological activity and/or properties of previously untested chemical compound of drug prior to experimental synthesis, thereby saving time, cost and efforts involved in traditional methods of drug design and discovery.

The processor 104 may include suitable logic, circuitry, and/or interfaces that is configured to respond and process the instructions required to drive the system 100A. Furthermore, the processor 104 may refer to one or more individual processors, processing devices and various elements associated with a processing device that may be shared by other processing devices. Additionally, the one or more individual processors, processing devices and elements are arranged in various architectures for responding to and processing the instructions to drive the system 100A. In an implementation, the processor 104 may be an independent unit and located outside the server 102 of the system 100A. Examples of the processor 104 may include, but are not limited to a hardware processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

The memory 106 may include suitable logic, circuitry, and/or interfaces that is configured to store data and the instructions executable by the processor 104. Examples of implementation of the memory 106 may include, but are not limited to, an Electrically Erasable Programmable Read-Only Memory (EEPROM), Random Access Memory (RAM), Read Only Memory (ROM), Hard Disk Drive (HDD), Flash memory, Solid-State Drive (SSD), or CPU cache memory. The memory 106 may store an operating system or other program products (including one or more operation algorithms) to operate the system 100A. In an embodiment, the memory 106 may be configured to store the pre-trained NLP model 108 and the deep learning model 110. The deep learning model 110 requires training to reliably predict the biological activities or properties of the one or more chemical compounds present in the new drug. Furthermore, the memory 106 is connected to the storage system 112 to receive the plurality of input datasets 114 comprising the plurality of SMILES notations 114A for processing by the processor 104.

The storage system 112 may include suitable logic, circuitry, and/or interfaces that is configured to store the plurality of input datasets 114. Examples of implementation of the storage system 112 are similar to examples of implementation of the memory 106. In an embodiment, the storage system 112 may be a database system that may be configured to store the plurality of SMILES notations 114A of the one or more chemical compounds. Generally, a SMILES notation is a line notation which describes a chemical structure of a chemical compound using short ASCII strings.

The communication network 118 may include suitable logic, circuitry, and/or interfaces through which the server 102 is connected to the user device 116. Examples of implementation of the communication network 118 may include, but are not limited to, a cellular network (e.g., a 2G, a 3G, long-term evolution (LTE) 4G, a 5G, or 5G NR network, such as sub 6 GHZ, cmWave, or mmWave communication network), a wireless sensor network (WSN), a cloud network, a Local Area Network (LAN), a vehicle-to-network (V2N) network, a Metropolitan Area Network (MAN), and/or the Internet.

The user device 116 may include suitable logic, circuitry, and/or interfaces that is used by a user (not shown in FIG. 1A) for evaluating the predicted biological activities or properties of the one or more chemical compounds present in the drug. The user device 116 comprises the user interface 120 which displays the predicted biological activities or properties of the one or more chemical compounds present in the drug to the user. Examples of implementation of the user device 116 may include, but are not limited to, a computer, mobile phone, laptop, a display device, and the like.

In operation, the aspects of the disclosed embodiments provide the system 100A for predicting biological activities or properties of one or more chemical compounds in a new drug. The system 100A comprises the processor 104 configured to receive the plurality of input datasets 114, wherein the plurality of input datasets 114 comprises the plurality of simplified molecular-input line-entry system (SMILES) notations 114A associated with a plurality of drugs, wherein each of the plurality of SMILES notations 114A is an indicative of a chemical structure of a chemical compound present in a drug of the plurality of drugs. The reliable prediction of the biological activities or properties (e.g., molecular properties, physiochemical properties, and the like) of the one or more chemical compounds present in the new drug leads to saving a lot of time, cost and efforts involved in the design and discovery of the new drug. The processor 104 of the server 102 of the system 100A is configured to receive the plurality of input datasets 114 comprising the plurality of SMILES notations 114A for processing. Each of the plurality of SMILES notations 114A indicates the chemical structure of the chemical compound present in the drug. The chemical structure may include a 3D chemical structure which represents how the various atoms are arranged in a real 3D space. In an implementation, one of the plurality of SMILES notations 114A may represent a newly generated chemical compound for which any biological activity or property is not predicted before. In said implementation scenario, the system 100A is used to predict the one or more biological activities or properties of the newly generated chemical compound by use of one of the plurality of SMILES notations 114A. In another implementation, one of the plurality of SMILES notations 114A may represent a previously generated chemical compound which has one or more biological activities or properties and therefore, using the system 100A, a verification of the one or more biological activities or properties of the previously generated chemical compound, can be obtained. Additionally, the plurality of input datasets 114 comprises the known biological activities or properties of one or more chemical compounds present in the new drug. The processor 104 is further configured to execute the pre-trained Natural Language Processing (NLP) model 108 to transform the plurality of SMILES notations 114A into a plurality of non-sparse matrices, wherein each of the plurality of non-sparse matrices is indicative of one or more biological activities of the chemical compound present in the drug. The pre-trained NLP model 108 is executed to transform the plurality of SMILES notations 114A into the plurality of non-sparse matrices. Each of the plurality of non-sparse matrices represents each and every feature of the chemical compound in each dimension including 1D, 2D and 3D space. Each of the plurality of non-sparse matrices includes lesser number of zeros in comparison to a conventional sparse matrix, which means each feature is unique in itself and has negligible similarity with another feature of the chemical compound.

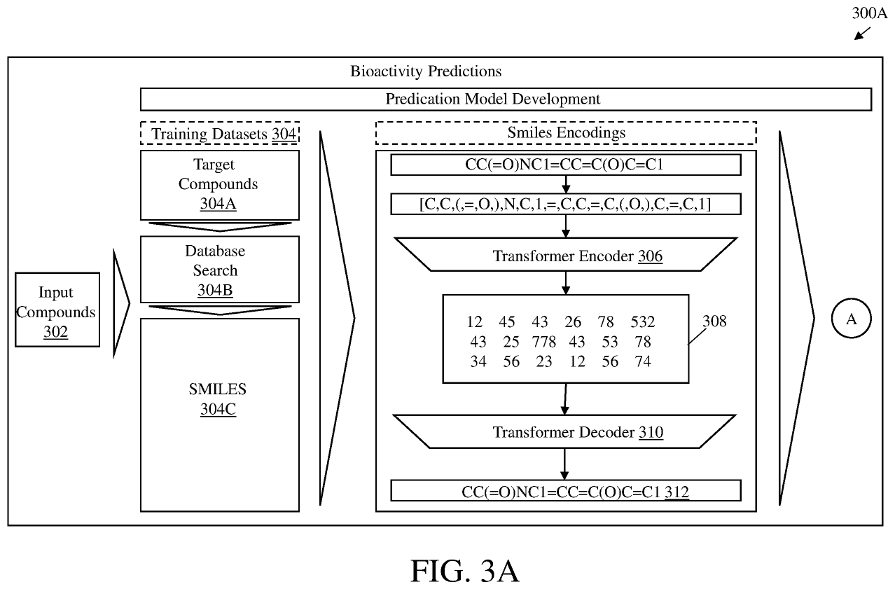

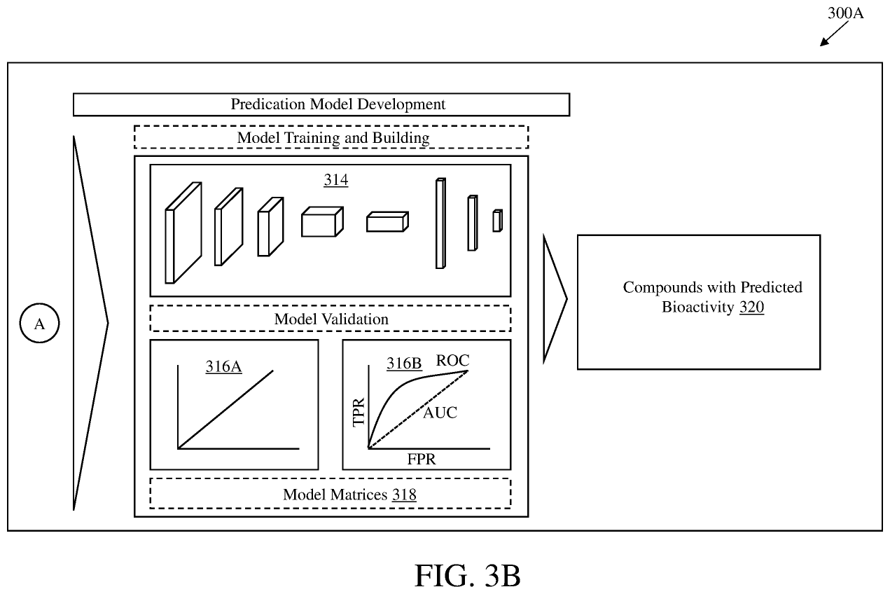

In accordance with an embodiment, transformation of the plurality of SMILES notations 114A into the plurality of non-sparse matrices comprises transforming each of the plurality of SMILES notations 114A into a corresponding plurality of word embeddings by splitting each of the plurality of SMILES notations 114A and transforming, via an encoder stack, the corresponding plurality of word embeddings of each of the plurality of SMILES notations 114A into a corresponding non-sparse matrix of the plurality of non-sparse matrices, wherein the pre-trained NLP model 108 comprises the encoder stack and a decoder stack. In an implementation, one of the plurality of SMILES notations 114A may be represented as C1=CC=CC=C1 which is splitted into the corresponding plurality of word embeddings that may be represented as C1, =, C, C, =, C, C, =, C1. Each chemical compound can be represented by the plurality of word embeddings and each of the plurality of word embeddings is a collection of strings. The splitting of each of the plurality of SMILES notations 114A into the corresponding plurality of word embeddings improves the understanding of each of the plurality of SMILES notations 114A as well as improves the accuracy of the pre-trained NLP model 108. Furthermore, the pre-trained NLP model 108 comprises the encoder stack and the decoder stack. The encoder stack is configured to convert the corresponding plurality of word embeddings of each of the plurality of SMILES notations 114A into the corresponding non-sparse matrix of the plurality of non-sparse matrices, shown in detail, for example, in FIG. 3A.

In accordance with an embodiment, the transformation of each of the plurality of SMILES notations 114A into the plurality of non-sparse matrices via the pre-trained NLP model 108 further comprises detecting one or more special characters present in each of the plurality of SMILES notations 114A. In each of the plurality of SMILES notations 114A, the 3D conformations (i.e., one or more spatial arrangements which the atoms in a molecule can adopt and freely convert between, specifically by rotation around individual single bonds) are addressed by the special characters (or notations), for example, @, # and branches by ( ) double bond configuration by /, and the like. In an implementation, the pre-trained NLP model 108 may be configured to detect the presence as well as removal of the one or more special characters from each of the plurality of SMILES notations 114A.

In accordance with an embodiment, the processor 104 is further configured to pre-process each of the plurality of SMILES notations 114A before executing the pre-trained NLP model 108 for the transformation of each of the plurality of SMILES notations 114A, wherein the pre-processing of each of the plurality of SMILES notations 114A comprises cleansing of each of the plurality of SMILES notations 114A by removal of an outer layer of salt in the chemical structure, detecting one or more outliers in each of the plurality of SMILES notations 114A using a cluster-based local outlier factor (CBLOF), canonicalizing each of the plurality of SMILES notations 114A and augmenting the canonicalized SMILES notations. Each of the plurality of SMILES notations 114A is pre-processed before transformation of each of the plurality of SMILES notations 114A into the corresponding plurality of word embeddings. Initially, each of the plurality of SMILES notations 114A is cleaned (or sanitized) by removal of the outer layer of salt in the chemical structure. The outer layer of salt is added to the chemical compound for stability of the chemical compound. Moreover, the outer layer of salt does not add any functional property to the chemical compound. In an implementation, each of the plurality of SMILES notations 114A may be cleaned by removal of other charged atoms by splitting each of the plurality of SMILES notations 114A into the corresponding plurality of word embeddings and retaining larger fragment. Thereafter, the one or more outliers (e.g., one or more anomalies) are detected in each of the plurality of SMILES notations 114A using the CBLOF method which is a cluster-based method. In CBLOF method, based on the features or properties (e.g., molecular structure), the one or more chemical compounds are grouped into one or more clusters and thereafter, it is detected that which chemical compounds with similar features or properties are falling into one cluster. The one or more chemical compounds with different properties (or different molecular structure) are not grouped into any cluster. After the anomaly detection, each of the plurality of SMILES notations 114A is canonicalized which means that each SMILES notation is represented in a standard format. In an implementation, each of the plurality of SMILES notations 114A may have isomeric SMILES and therefore, each of the plurality of SMILES notations 114A including the isomeric SMILES are converted into canonical form. Each of the plurality of SMILES notations 114A may be canonicalized using a RDkit, which is an open-source cheminformatics software and used for representing each of the plurality of SMILES notations 114A into canonicalized SMILES notations. Thereafter, each of the canonicalized SMILES notations is augmented using the RDkit. That means the pre-trained NLP model 108 is subjected to different number of iterations to train the model and to check whether the pre-trained NLP model 108 is ready to transform each of the plurality of SMILES notations 114A.

In accordance with an embodiment, each of the plurality of non-sparse matrices is a matrix of dimensions N×1024, wherein N is number of characters present in each of the plurality of SMILES notations 114A. Each of the plurality of non-sparse matrices is a numeric vector input that represents each character (or letter) of each of the plurality of SMILES notations 114A in a lower dimensional space. Furthermore, each non-sparse matrix allows the same letters to have a similar representation. Each of the plurality of SMILES notations 114A is useful to reduce the dimensionality and understand the types of bonds between atoms and inter-atomic semantics which is further used to represent the atoms in canonical SMILES format.

In accordance with an embodiment, the pre-trained NLP model 108 is a transformer model. The transformer model is used to solve sequence-to-sequence tasks while handling long-range dependencies with case. The transformer model is a transduction model relying entirely on self-attention to compute representations of input and output without using sequence-aligned recurrent neural network (RNN) or convolution. Transduction means the conversion of input sequences into output sequences. The transformer model is used to handle the dependencies between input and output with attention and recurrence completely. The pre-trained NLP model 108 may also be referred to as a scalable model.

In accordance with an embodiment, the encoder stack and the decoder stack of the pre-trained NLP model 108 comprises same number of encoders and decoders. The encoder stack and the decoder stack of the pre-trained NLP model 108 comprises multiple identical encoders and decoders, respectively, stacked on top of each other. The number of encoders in the encoder stack and the decoders in the decoder stack is a hyperparameter. In an implementation, for training of the pre-trained NLP model 108, eight encoders and decoders may be used.

In accordance with an embodiment, one encoder block from the encoder stack comprises a layer of multi-head attention followed by another layer of a feed forward neural network, and wherein one decoder block from the decoder stack comprises a single layer of masked multi-head attention. The encoder stack and the decoder stack of the pre-trained NLP model 108 work in the following way: each of the plurality of word embeddings of an input sequence (e.g., an individual SMILES notation) is passed to a first encoder in the encoder stack. The plurality of word embeddings is then transformed by the first encoder and propagated to a next encoder in the encoder stack. This way, an output from a last encoder in the encoder stack is passed to all the decoders in the decoder stack where the output from the encoder stack is decoded. Moreover, the layer of multi-head attention comprised by each encoder block of the encoder stack is computed by using self-attention, which is calculated in parallel and independently multiple times across the transformer's architecture and the outputs are concatenated and linearly transformed.

The processor 104 is further configured to train the deep learning model 110 using the plurality of non-sparse matrices to obtain a trained deep learning model, wherein the trained deep learning model is used to predict one or more biological activities of an untested chemical compound in the new drug when the chemical compound in the drug is subjected to at least one chemical modification to form the new drug. After transformation of the plurality of SMILES notations 114A into the plurality of non-sparse matrices, the processor 104 is configured to train the deep learning model 110. In an implementation, the deep learning model 110 may be trained using small molecular databases like chemical database of bioactive molecules with drug-like properties (i.e., ChEMBL) or PubChem Bioassay. The PubChem is public repository for information on chemical substances and their biological activities. The training of the deep learning model 110 using the small molecular databases enhances the process of discovery of a new chemical compound via the predictions of one or more biological activities or properties including physiochemical pharmacokinetic or toxicological properties. As the new chemical compound progresses down the development pipeline, the trained deep learning model may be used to predict the one or more biological activities or properties of the new chemical compound in order to reduce cost, time and to prevent development failures. In another implementation, when the chemical compound present in the drug is subjected to at least one chemical change to form the new drug, results in generation of the untested chemical compound. And, the trained deep learning model is used to predict the one or more biological activities of the untested chemical compound in the new drug.

In accordance with an embodiment, the at least one chemical modification of the chemical compound comprises alteration of one or more functional groups, an atomic level variation, a change in a bond connecting an atomic pair, or any other modification in the chemical structure of the chemical compound in the drug to form the new drug. In an implementation, the new drug can be formed by alteration of the one or functional groups in the chemical compound already present in the drug. Generally, a functional group is defined as an atom or a group of atoms which is joined in a specific manner and responsible for chemical properties of the chemical compound. Examples of the functional group may include, but are not limited to, a hydroxyl group (—OH), an aldehyde group (—CHO), ketonic group (—CO—), and the like. In another implementation, the new drug can be formed due to the atomic level variation that is by varying the size, shape and electronic distribution of atoms. In a yet another implementation, the new drug can be formed by doing the change in the bond connecting the atomic pair or the other modification in the chemical structure of the chemical compound already present in the drug.

In accordance with an embodiment, the processor 104 is further configured to split the plurality of input datasets 114 into a training dataset, a validation dataset and a test dataset. In an implementation scenario, the plurality of input datasets 114 may be splitted into the training dataset ranging from 1 to 70%, the validation dataset ranging from 71 to 85% and the test dataset ranging from 86 to 100% of the plurality of input datasets 114. In another implementation scenario, the plurality of input datasets 114 may be splitted into the training dataset ranging from 1 to 75%, the validation dataset ranging from 76 to 85% and the test dataset ranging from 86 to 100% of the plurality of input datasets 114.

In accordance with an embodiment, the processor 104 is further configured to train the deep learning model 110 from the plurality of non-sparse matrices by passing each of the plurality of non-sparse matrices into a first one-dimensional convolution layer to calculate a first set of output vectors and processing at least one datapoint in each vector of the first set of output vectors using a second one-dimensional convolution layer to calculate a second set of output vectors. In an implementation, the deep learning model 110 may use a convolutional neural network (CNN) that comprises a stack of convolution layers followed by pooling layers and another stack of fully connected layers. The deep learning model 110 is trained by passing each of the plurality of non-sparse matrices into the first one-dimensional convolution layer with a kernel size of 5, which computes the first set of output vectors. The first set of output vectors is further passed into a first one-dimensional max pooling layer, which computes a maximum value in each vector of the first set of output vectors. The results obtained from the first one-dimensional max pooling layer are down-sampled and the pooled feature maps which potentially represent the first set of output vectors are considered. Thereafter, the maximum value (i.e., the at least one datapoint) in each vector of the first set of output vectors is passed into the second one-dimensional convolution layer with a kernel size of 3, which computes the second set of output vectors. The second set of output vectors are passed into a second one-dimensional max pooling layer, which computes a maximum value in each vector of the second set of output vectors. Thereafter, the maximum value in each vector of the second set of output vectors is passed into the other stack of fully connected layers of reducing unit sizes of 512, 256, 64 and 16 to reduce the dimensionality of second set of output vectors. On the basis of dimensions of the prediction label or value, the second set of output vectors is passed into a dense layer with an activation function of rectified linear unit (ReLU) to obtain a resultant dataset of reduced dimensions. The activation function of ReLU is used for regression and sigmoid for binary classification.

Moreover, in some cases, in order to reduce the problem of overfitting of the training dataset, a dropout of cut-off 0.05 is applied on the training dataset. Generally, dropout is a regularization method that approximates training a large number of neural networks with different architectures in parallel.

In accordance with an embodiment, the trained deep learning model is validated using a combination of regression scores, a mean square error, and a root mean square error applied on the second set of output vectors. After training of the deep learning model 110, the trained deep learning model is obtained, which is validated using validation matrices of the regression scores (r²), the mean square error (MSE) and the root mean square error (RMSE) applied on the second set of output vectors. The validation matrices of r², MSE and RMSE is used for regression type datasets. Another validation matrices of model accuracy and receiver operating curve (ROC curve) is used for binary datasets. In order to further validate the reliability of the trained deep learning model on the basis of an input dataset, a ten-fold cross validation of the trained deep learning model may be carried out on a ten-fold data split.

In accordance with an embodiment, the prediction of the one or more biological activities of the untested chemical compound comprises prediction of one or more biological activity constants, wherein the one or more biological activity constants include at least one of: a half-maximal inhibitory concentration (IC50), a 90% inhibitory concentration (IC90), a dissociation constant (Kd), a half maximal effective concentration (EC50), and a lethal dose to 50% (LD50). In addition to prediction of the one or more biological activities of the untested chemical compound, the trained deep learning model is used to predict the one or more biological activity constants, such as the half-maximal inhibitory concentration (IC50), the 90% inhibitory concentration (IC90), the dissociation constant (Kd), the half maximal effective concentration (EC50), and the lethal dose to 50% (LD50). Generally, the half-maximal inhibitory concentration (IC50) is widely used to measure a drug's efficacy. The IC50 indicates how much drug is required to inhibit a biological process by half, thus providing a measure of potency of an antagonist drug in pharmacological research. The half maximal effective concentration (EC50) indicates concentration of a drug that is required to cause half of the maximum possible effect. The prediction of the one or more biological activity constants of the untested chemical compound leads to saving time, cost and efforts involved in design and discovery of the new drug.

In accordance with an embodiment, the processor 104 is further configured to determine one or more physiochemical properties of the untested chemical compound via the trained deep learning model, wherein the one or more physiochemical properties comprises one or more of: a melting point, a boiling point, a solubility, and an Absorption, Distribution, Metabolism, Excretion, and Toxicity (ADMET) properties of the untested chemical compound. The trained deep learning model is further configured to determine the one or more physiochemical properties, such as the melting point, the boiling point, the solubility and the ADMET properties of the untested chemical compound.

Thus, the system 100A efficiently and reliably predicts the biological activities or properties of the chemical compound already present in the drug by virtue of using the pre-trained NLP model 108 and training of the deep learning model 110. The prediction of the biological activities and the one or more physiochemical properties (e.g., the ADMET properties, and the like) of the chemical compound in the drug with improved accuracy not only reduces the cost of the drug discovery but also has a direct positive impact on an individual's health. For example, if the bioactivity of a potential drug is estimated in advance, then it can be decided whether the drug can be selected for further studies or not, thereby saving cost, time and efforts. Since the one or more biological activities or properties of the chemical compound are diverse in comparison to the structural properties, drawing a reliable relation between the one or more biological activities and the structural properties of the chemical compound is a tedious task. On the contrary, the system 100A with the pre-trained NLP model 108 and the deep learning model 110, manifests an improved prediction accuracy and hence, draws the reliable relation between the one or more biological activities and the structural properties of the chemical compound, further makes the process of drug development more reliable. Conventionally, a drug is biased for use in a particular sale line only and the drug is found as a failure when used in a different sale line. In contrast to the conventional drug, the system 100A removes the biasness of the drug and on experimentation, the drug is found to be applicable for use in different sale lines.

FIG. 1B is a block diagram of a system of predicting biological activities or properties of one or more chemical compounds in a new drug, in accordance with another embodiment of the present disclosure. FIG. 1B is described in conjunction with elements from FIG. 1A. With reference to FIG. 1B, there is shown a block diagram of a system 100B that includes a trained deep learning model 122.

The system 100B is similar to the system 100A (of FIG. 1A) except that the system 100A includes training of the deep learning model 110 to obtain a trained deep learning model and the system 100B includes execution of the trained deep learning model 122. Therefore, the system 100B is applicable to predict one or more biological activities or properties of an untested chemical compound present in the new drug. Alternatively stated, the system 100B is advanced version of the system 100A.

In operation, the aspects of the disclosed embodiments provide the system 100B of predicting biological activities or properties of one or more chemical compounds in a new drug. The system 100B comprises the processor 104 configured to receive a simplified molecular-input line-entry system (SMILES) notation 124, wherein the SMILES notation 124 is an indicative of a chemical structure of an untested chemical compound present in the new drug. The SMILES notation 124 indicates the chemical structure of the untested chemical compound whose biological activities or properties are neither predicted nor tested before.

The processor 104 is further configured to execute the pre-trained Natural Language Processing (NLP) model 108 to transform the SMILES notation 124 into a non-sparse matrix. The SMILES notation 124 is processed into a plurality of word embeddings which is further transformed by the encoder stack of the pre-trained NLP model 108 into the non-sparse matrix.

The processor 104 is further configured to execute the trained deep learning model 122 by passing the non-sparse matrix to the trained deep learning model 122 to predict one or more biological activities and one or more physiochemical properties of the untested chemical compound in the new drug. The non-sparse matrix is used as an input to the trained deep learning model 122 which uses a stack of convolution layers and another stack of densely connected layers to obtain a resultant output dataset with reduced dimensionality. The resultant output dataset corresponds to the untested chemical compound with predicted one or more biological activities and one or more physiochemical properties. The resultant dataset is validated using validation matrices of regression scores (r²), MSE, RMSE, and the like.

The system 100B efficiently and reliably predicts the biological activities or properties of the untested chemical compound when the chemical compound already present in the drug is subjected to a change (e.g., 1% change in one of the structural features) to form the new drug by virtue of using the pre-trained NLP model 108 and the trained deep learning model 122. Alternatively stated, Conventionally, structural similarity between chemical compounds was used to predict certain limited biological activities of chemical compositions present in drug. Suppose a novel drug exhibits 2% change in chemical composition then, by use of structural similarity it becomes difficult and technically challenging to predict any change in biological properties of one or more chemical compounds present in a new or novel drug. In the present disclosure, instead of structural similarity, a non-sparse matrix is generated by the system 100B using a transformer model (i.e., the pre-trained NLP model 108), which is then used to train the deep learning model to predict biological activity and/or properties of previously untested chemical compound of drug prior to experimental synthesis, thereby saving time, cost and efforts involved in traditional methods of drug design and discovery.

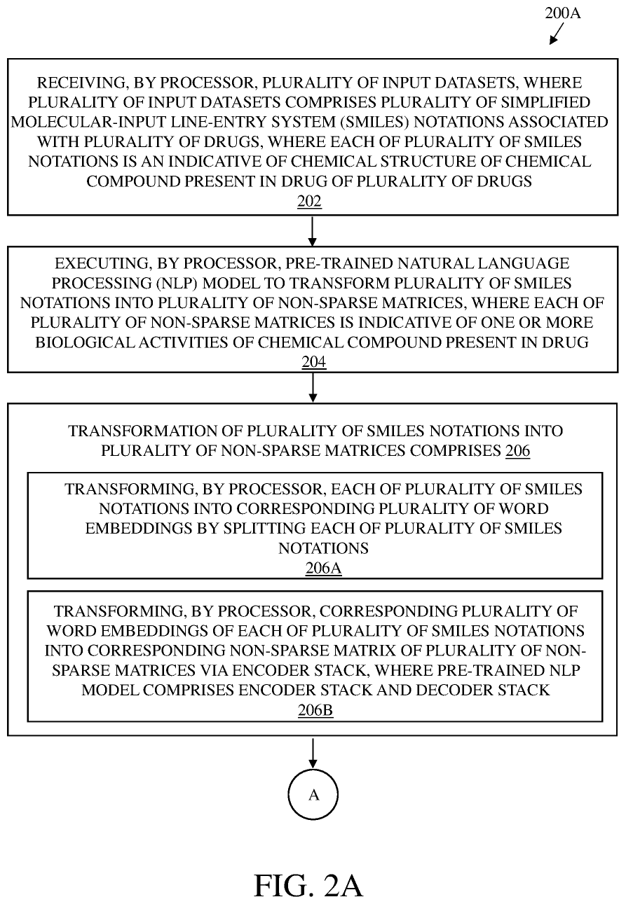

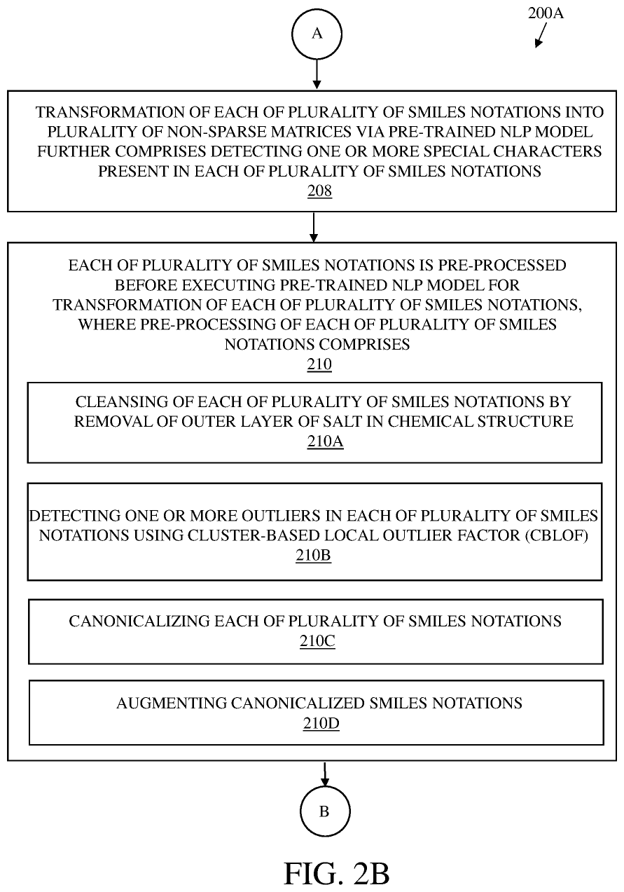

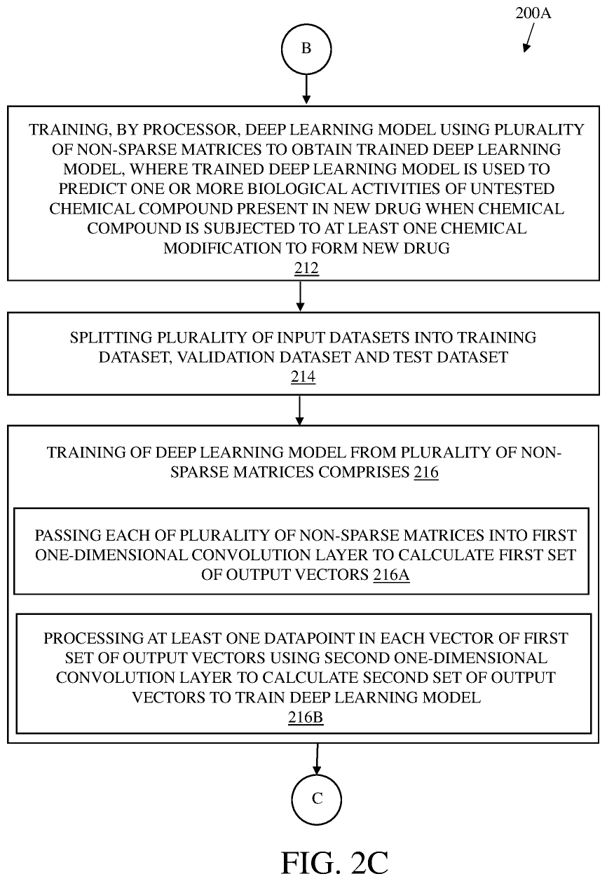

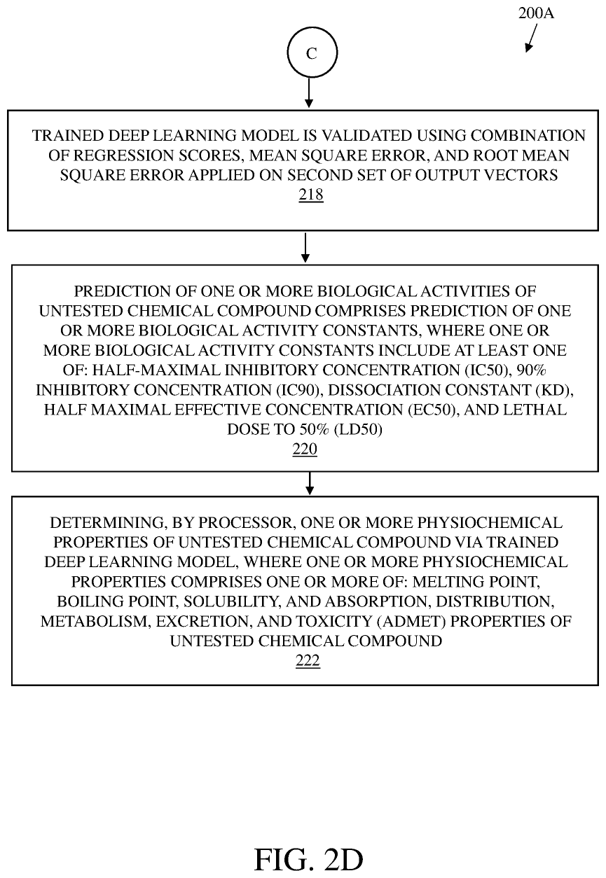

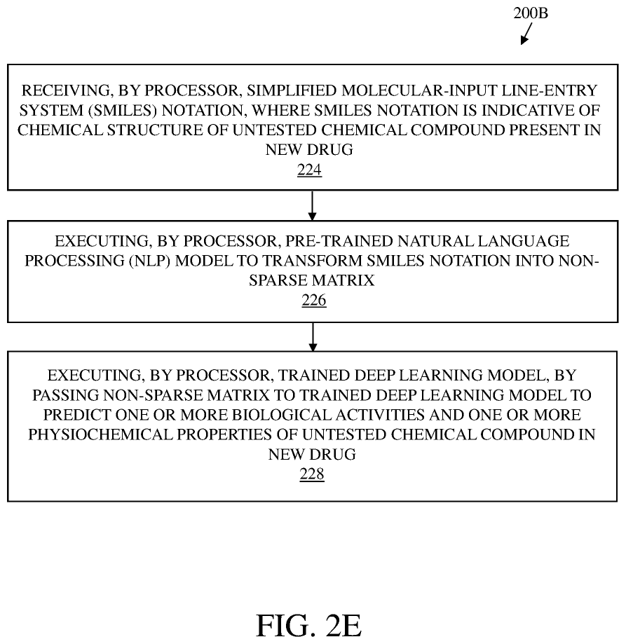

FIGS. 2A to 2D collectively is a flow chart of a method of predicting biological activities or properties of one or more chemical compounds in a new drug, in accordance with an embodiment of the present disclosure. FIGS. 2A-2D are described in conjunction with elements from FIG. 1A. With reference to FIGS. 2A to 2D, there is shown a flowchart of a method 200A that includes steps 202, 204, 206, 208, 210, 212, 214, 216, 218, 220, and 222. The step 206 includes sub steps 206A and 206B, the step 210 includes sub steps 210A. 210B. 210C, and 210D, and the step 216 includes sub steps 216A and 216B. The steps 202 to 206 (including sub steps 206A and 206B) are shown in FIG. 2A, the steps 208 to 210 (including sub steps 210A, 210B, 210C and 210D) are shown in FIG. 2B, the steps 212 to 216 (including sub steps 216A and 216B) are shown in FIG. 2C, and the steps 218 to 222 are shown in FIG. 2D. The method 200A is executed by the processor 104 of the system 100A (of FIG. 1A).

There is provided the method 200A of predicting biological activities or properties of one or more chemical compounds in a new drug.

At step 202, the method 200A comprises receiving, by the processor 104, the plurality of input datasets 114, where the plurality of input datasets 114 comprises the plurality of simplified molecular-input line-entry system (SMILES) notations 114A associated with a plurality of drugs, wherein each of the plurality of SMILES notations 114A is an indicative of a chemical structure of a chemical compound present in a drug of the plurality of drugs.

At step 204, the method 200A further comprises executing, by the processor 104, the pre-trained Natural Language Processing (NLP) model 108 to transform the plurality of SMILES notations 114A into the plurality of non-sparse matrices, wherein each of the plurality of non-sparse matrices is indicative of one or more biological activities of the chemical compound present in the drug. Each of the plurality of non-sparse matrices is a matrix of dimensions N×1024, wherein N is number of characters present in each of the plurality of SMILES notations 114A. Moreover, the pre-trained NLP model 108 is a transformer model.

At step 206, transformation of the plurality of SMILES notations 114A into the plurality of non-sparse matrices is performed in two sub steps 206A and 206B.

At sub step 206A of the step 206, the method 200A further comprises transforming, by the processor 104, each of the plurality of SMILES notations 114A into a corresponding plurality of word embeddings by splitting each of the plurality of SMILES notations 114A.

At sub step 206B of the step 206, the method 200A further comprises transforming, by the processor 104, the corresponding plurality of word embeddings of each of the plurality of SMILES notations 114A into a corresponding non-sparse matrix of the plurality of non-sparse matrices via an encoder stack, wherein the pre-trained NLP model 108 comprises the encoder stack and a decoder stack. The encoder stack and the decoder stack of the pre-trained NLP model 108 comprises same number of encoders and decoders. Furthermore, one encoder block from the encoder stack comprises a layer of multi-head attention followed by another layer of a feed forward neural network, and wherein one decoder block from the decoder stack comprises a single layer of masked multi-head attention.

Now referring to FIG. 2B, at step 208, the transformation of each of the plurality of SMILES notations 114A into the plurality of non-sparse matrices via the pre-trained NLP model 108 further comprises detecting one or more special characters present in each of the plurality of SMILES notations 114A.

At step 210, each of the plurality of SMILES notations 114A is pre-processed before executing the pre-trained NLP model 108 for the transformation of each of the plurality of SMILES notations 114A, wherein the pre-processing of each of the plurality of SMILES notations 114A is performed in four sub steps 210A, 210B, 210C, and 210D.

At sub step 210A of the step 210, the pre-processing of each of the plurality of SMILES notations 114A comprises cleansing of each of the plurality of SMILES notations 114A by removal of an outer layer of salt in the chemical structure.

At sub step 210B of the step 210, the pre-processing of each of the plurality of SMILES notations 114A further comprises detecting one or more outliers in each of the plurality of SMILES notations 114A using a cluster-based local outlier factor (CBLOF).

At sub step 210C of the step 210, the pre-processing of each of the plurality of SMILES notations 114A further comprises canonicalizing each of the plurality of SMILES notations 114A.

At sub step 210D of the step 210, the pre-processing of each of the plurality of SMILES notations 114A further comprises augmenting the canonicalized SMILES notations.

Now referring to FIG. 2C, at step 212, the method 200A further comprises training, by the processor 104, the deep learning model 110 using the plurality of non-sparse matrices to obtain a trained deep learning model, wherein the trained deep learning model is used to predict one or more biological activities of an untested chemical compound present in the new drug when the chemical compound is subjected to at least one chemical modification to form the new drug.

Optionally, the at least one chemical modification of the chemical compound comprises alteration of one or more functional groups, an atomic level variation, a change in a bond connecting an atomic pair, or any other modification in the chemical structure of the chemical compound in the drug to form the new drug.

At step 214, the method 200A further comprises splitting the plurality of input datasets 114 into a training dataset, a validation dataset and a test dataset.

At step 216, the training of the deep learning model 110 from the plurality of non-sparse matrices is performed in two sub steps 216A and 216B.

At sub step 216A of the step 216, the training of the deep learning model 110 from the plurality of non-sparse matrices comprises passing each of the plurality of non-sparse matrices into a first one-dimensional convolution layer to calculate a first set of output vectors.

At sub step 216B of the step 216, the training of the deep learning model 110 from the plurality of non-sparse matrices further comprises processing at least one datapoint in each vector of the first set of output vectors using a second one-dimensional convolution layer to calculate a second set of output vectors to train the deep learning model 110.

Now referring to FIG. 2D, at step 218, the trained deep learning model is validated using a combination of regression scores, a mean square error, and a root mean square error applied on the second set of output vectors.

At step 220, the prediction of the one or more biological activities of the untested chemical compound comprises prediction of one or more biological activity constants, wherein the one or more biological activity constants include at least one of: a half-maximal inhibitory concentration (IC50), a 90% inhibitory concentration (IC90), a dissociation constant (Kd), a half maximal effective concentration (EC50), and a lethal dose to 50% (LD50).

At step 222, the method 200A further comprises determining, by the processor 104, one or more physiochemical properties of the untested chemical compound via the trained deep learning model, wherein the one or more physiochemical properties comprises one or more of: a melting point, a boiling point, a solubility, and an Absorption, Distribution, Metabolism, Excretion, and Toxicity (ADMET) properties of the untested chemical compound.

The steps 202 to 222 are only illustrative and other alternatives can also be provided where one or more steps are added, one or more steps are removed, or one or more steps are provided in a different sequence without departing from the scope of the claims herein.

FIG. 2E is a flowchart of a method of predicting biological activities or properties of one or more chemical compounds in a new drug, in accordance with an embodiment of the present disclosure. FIG. 2E is described in conjunction with elements from FIGS. 1A, 1B, and 2A-2D. With reference to FIG. 2E, there is shown a method 200B that includes steps 224, 226, and 228. The method 200B is executed by the processor 104 of the system 100B (of FIG. 1B).

There is provided the method 200B of predicting biological activities or properties of one or more chemical compounds in a new drug.

At step 224, the method 200B comprises receiving, by the processor 104, the simplified molecular-input line-entry system (SMILES) notation 124, wherein the SMILES notation 124 is an indicative of a chemical structure of an untested chemical compound present in the new drug.

At step 226, the method 200B further comprises executing, by the processor 104, the pre-trained Natural Language Processing (NLP) model 108 to transform the SMILES notation 124 into a non-sparse matrix.

At step 228, the method 200B further comprises executing, by the processor 104, the trained deep learning model 122, by passing the non-sparse matrix to the trained deep learning model 122 to predict one or more biological activities and one or more physiochemical properties of the untested chemical compound in the new drug.

The steps 224 to 228 are only illustrative and other alternatives can also be provided where one or more steps are added, one or more steps are removed, or one or more steps are provided in a different sequence without departing from the scope of the claims herein.

FIGS. 3A-3B collectively illustrate how to predict bioactivity of one or more chemical compounds present in a drug, in accordance with an embodiment of the present disclosure. FIGS. 3A-3B are described in conjunction with elements from FIGS. 1A, 1B, 2A-2D, and 2E. With reference to FIGS. 3A-3B, there is shown development of a prediction model 300A.

Referring to FIG. 3A, there is shown a plurality of input chemical compounds 302, training datasets 304, a transformer encoder 306, a non-sparse matrix 308 and a transformer decoder 310.

Each of the plurality of input chemical compounds 302 has their respective identity and SMILES notation, as represented in a Table 1.

TABLE 1

| Identity (ID) | SMILES |
|---|---|
| SMILES1 | CNC(═O)C1(c2c . . . |
| SMILES2 | COc1ccc2c(c1)C(. . . |
| SMILES3 | CCN1CCN(C(═O . . . |

The training datasets 304 includes a plurality of target compounds 304A, such as protein, cell line and Bioassay, a database search 304B and a plurality of SMILES notations 304C of each of the plurality of input chemical compounds 302. The database search 304B includes PubChem and ChEMBL molecular databases. The plurality of SMILES notations 304C of each of the plurality of input chemical compounds 302 with bioactivity measured in nano metres (nm) is represented in a Table 2.

TABLE 2

| SMILES | Activity (nm) |
|---|---|
| CNC(═O)C1(c2c . . . | 43 |
| COc1ccc2c(c1)C(. . . | 678 |
| CCN1CCN(C(═O . . . | 44 |
| COC(═O)CC(c1c . . . | 55 |
| COc1cccc2c1ncc1 | 7000 |
| CCC(C)n1c(═O)c2 | 45235 |

Thereafter, each of the plurality of SMILES notations 304C is transformed into a plurality of word embeddings. For example, a SMILES notation may be represented as CC(=O)NC1=CC=C(O)C=C1 and its corresponding plurality of word embeddings may be represented as [‘C’, ‘C’, ’(’, ‘=’, ‘O’, ‘)’, ‘=’, ‘N’, ‘C’, ‘1’, ‘=’, ‘C’, ‘C’, ‘=’, ‘(’, ‘O’, ‘)’, ‘C’, ‘=’, ‘C’, ‘1’]. The plurality of word embeddings in transformed into a non-sparse matrix 308 by use of the transformer encoder 306 (i.e., the encoder stack of the pre-trained NLP model 108). The non-sparse matrix 308 may be represented as

$$\\ [ \begin{matrix}12 & 45 & 43 & 26 & 78 & 532 & ⋯ \\ 43 & 25 & 778 & 43 & 53 & 78 & ⋯ \\ 34 & 56 & 23 & 12 & 56 & 74 & ⋯ \\ 342 & 54 & 23 & 5 & 7 & 423 & ⋯ \\ ⋯ & ⋯ & ⋯ & ⋯ & ⋯ & ⋯ & ⋯\end{matrix} ]$$

The non-sparse matrix 308 is decoded by use of the transformer decoder 310 (i.e., the decoder stack of the pre-trained NLP model 108) to obtain a modified SMILES notation 312, which may be represented as CC(=O)NC1=CC=C(O)C=C1.

Now referring to FIG. 3B, in FIG. 3B, training and validation of the deep learning model 110 (of FIG. 1A) is shown. The training of the deep learning model 110 is performed by using a convolutional neural network 314 to obtain the trained deep learning model 122 (of FIG. 1B). Furthermore, the trained deep learning model 122 is validated using a regression plot 316A and a ROC-AUC curve 316B (i.e., area under the ROC curve) and a plurality of validation matrices 318 including MSE, and RMSE matrices. Finally, a plurality of output chemical compounds 320 is obtained with predicted bioactivity.

The convolutional neural network 314 comprises a stack of convolution layers followed by pooling layers and another stack of fully connected layers. The deep learning model 110 is trained by passing the non-sparse matrix 308 into the stack of convolution layers followed by pooling layers (e.g., max pooling layers) and then, the stack of fully connected layers, have been described in detail, for example, in FIG. 1A. The trained deep learning model 122 provides improved predictions of bioactivities on the plurality of target compounds 304A by easily quantifying the properties of the plurality of input chemical compounds 302 at the atomic level. Thereafter, the trained deep learning model 122 is validated using the plurality of validation matrices 318 (or model matrices) that includes RMSE and regression scores (r²), and accuracy and F1 score. After validation of the trained deep learning model 122, the plurality of output chemical compounds 320 is obtained with predicted bioactivity, represented in Table 3.

TABLE 3

| Identity (ID) | SMILES | IC50 (nm) |
|---|---|---|
| SMILES1 | CNC(═O)C1(c2c . . . | 20 |
| SMILES2 | COc1ccc2c(c1)C(. . . | 149 |
| SMILES3 | CCN1CCN(C(═O . . . | 5000 |

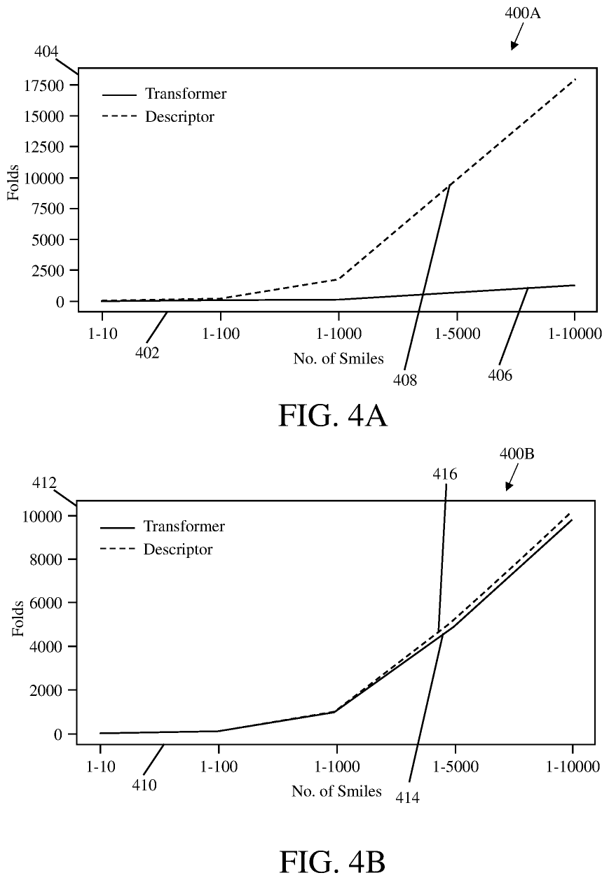

FIG. 4A is a graphical representation that illustrates comparison of time complexity between a transformer model and a conventional PaDEL-descriptor model, in accordance with an embodiment of the present disclosure. FIG. 4A is described in conjunction with elements from FIGS. 1A-1B, 2A-2D, 2E, and 3A-3B. With reference to FIG. 4A, there is shown a graphical representation 400A with an X-axis 402 that represents number of SMILES and a Y-axis 404 that represents number of folds.

In the graphical representation 400A, there is shown a first line 406 and a second line 408. The first line 406 represents the time complexity obtained by use of the transformer model (i.e., the pre-trained NLP model 108 of FIGS. 1A and 1B). The second line 408 represents the time complexity obtained by use of the conventional PaDEL-descriptor model. The PaDEL-descriptor is a software for calculating molecular descriptors and fingerprints. The transformer model comprises non-sparse embeddings whereas the conventional PaDEL-descriptor model comprises sparse embeddings. Specifically, a number of SMILES of drugs are considered as inputs to the transformer model and the PaDEL-descriptor model. The transformer model (i.e., the sequence-to-sequence model) converts the SMILES into a plurality of word embeddings which are further transformed into a non-sparse matrix. The non-sparse matrix is a numeric vector input that represents letters of the SMILES in a lower-dimensional space. Moreover, the graphical representation 400A illustrates that the time complexity of the transformer model (represented by the first line 406) is approximately 700 folds lower than that of the conventional PaDEL-descriptor model (represented by the second line 408). Additionally, the transformer model reduces dimensionality of an input data (i.e., input SMILES) and enables an efficient way to execute a machine learning model (e.g., the trained deep learning model 122) on large inputs resulting into less memory utilization as well.

FIG. 4B is a graphical representation that illustrates comparison of space complexity between a transformer model and a conventional PaDEL-descriptor model, in accordance with an embodiment of the present disclosure. FIG. 4B is described in conjunction with elements from FIGS. 1A-1B, 2A-2D, 2E, 3A-3B, and 4A. With reference to FIG. 4B, there is shown a graphical representation 400B with an X-axis 410 that represents number of SMILES and a Y-axis 412 that represents number of folds.

In the graphical representation 400B, there is shown a first line 414 and a second line 416. The first line 414 represents the space complexity obtained by use of the transformer model (i.e., the pre-trained NLP model 108 of FIGS. 1A and 1B). The second line 416 represents the space complexity obtained by use of the conventional PaDEL-descriptor model. The graphical representation 400B illustrates that the space complexity of the transformer model (represented by the first line 414) is approximately 43 folds lower than that of the conventional PaDEL-descriptor model (represented by the second line 416).

In addition to the conventional PaDEL-descriptor model, the performance of the transformer model (i.e., the pre-trained NLP model 108) is compared to other models, namely, ST along with Multi-layer Perceptron (i.e., ST+MLP), Extended-Connectivity Fingerprints (ECFP) Along with MLP (i.e., ECFP+MLP), Recurrent Neural Network (RNNS2S) along with MLP (i.e., RNNS2S+MLP), Graphical Convolution (i.e., GraphConv). The comparison is performed on basis of various parameters, such as Blood-Brain-Barrier Permeability (BBBP), Free Solvation (FreeSolv) and Estimated Solubility (ESOL). The performance comparison between aforementioned models and the transformer model is shown in Table 4:

TABLE 4
Performance evaluation metrics

| ROC AUC/RMSE | BBBP | FreeSolv | ESOL |
|---|---|---|---|
| ST + MLP | 0.9 | 2.246 | 1.144 |
| ECFP + MLP | 0.76 | 3.043 | 1.741 |
| RNNS2S + MLP | 0.884 | 2.987 | 1.317 |
| GraphConv | 0.795 | 3.476 | 1.673 |
| Transformer Model | 0.816 | 1.548 | 0.623 |

In order to compare aforementioned models and the transformer model, one classification dataset BBBP and two regression datasets namely, FreeSolv and ESOL are considered. For classification dataset, the ROC-AUC value is used as the performance metric and for the regression dataset, the RMSE value is used as the performance metric in the Table 4. From the Table 4, this can be concluded that the transformer model is more efficient and reliable and hence, can be used to develop further models for other similar datasets related to the drug design and discovery.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A method of predicting biological activities or properties of one or more chemical compounds in a new drug, the method comprising:
receiving, by a processor, a plurality of input datasets, wherein the plurality of input datasets comprises a plurality of simplified molecular-input line-entry system (SMILES) notations associated with a plurality of drugs, wherein each of the plurality of SMILES notations is an indicative of a chemical structure of a chemical compound present in a drug of the plurality of drugs;
executing, by the processor, a pre-trained Natural Language Processing (NLP) model to transform the plurality of SMILES notations into a plurality of non-sparse matrices, wherein each of the plurality of non-sparse matrices is indicative of one or more biological activities of the chemical compound present in the drug; and
training, by the processor, a deep learning model using the plurality of non-sparse matrices to obtain a trained deep learning model, wherein the trained deep learning model is used to predict one or more biological activities of an untested chemical compound present in the new drug when the chemical compound is subjected to at least one chemical modification to form the new drug.

2. The method according to claim 1, wherein transformation of the plurality of SMILES notations into the plurality of non-sparse matrices comprises:
transforming, by the processor, each of the plurality of SMILES notations into a corresponding plurality of word embeddings by splitting each of the plurality of SMILES notations; and
transforming, by the processor, the corresponding plurality of word embeddings of each of the plurality of SMILES notations into a corresponding non-sparse matrix of the plurality of non-sparse matrices via an encoder stack, wherein the pre-trained NLP model comprises the encoder stack and a decoder stack.

3. The method according to claim 2, wherein the encoder stack and the decoder stack of the pre-trained NLP model comprises a same number of encoders and decoders.

4. The method according to claim 3, wherein one encoder block from the encoder stack comprises a layer of multi-head attention followed by another layer of a feed forward neural network, and wherein one decoder block from the decoder stack comprises a single layer of masked multi-head attention.

5. The method according to claim 1, wherein each of the plurality of non-sparse matrices is a matrix of dimensions N×1024, wherein N is number of characters present in each of the plurality of SMILES notations.

6. The method according to claim 1, wherein the method further comprises splitting the plurality of input datasets into a training dataset, a validation dataset and a test dataset.

7. The method according to claim 1, wherein the training of the deep learning model from the plurality of non-sparse matrices comprises:
passing each of the plurality of non-sparse matrices into a first one-dimensional convolution layer to calculate a first set of output vectors; and
processing at least one datapoint in each vector of the first set of output vectors using a second one-dimensional convolution layer to calculate a second set of output vectors to train the deep learning model.

8. The method according to claim 7, wherein the trained deep learning model is validated using a combination of regression scores, a mean square error, and a root mean square error applied on the second set of output vectors.

9. The method according to claim 1, wherein the prediction of the one or more biological activities of the untested chemical compound comprises prediction of one or more biological activity constants, wherein the one or more biological activity constants include at least one of: a half-maximal inhibitory concentration (IC50), a 90% inhibitory concentration (IC90), a dissociation constant (Kd), a half maximal effective concentration (EC50), and a lethal dose to 50% (LD50).

10. The method according to claim 1, further comprising determining, by the processor, one or more physiochemical properties of the untested chemical compound via the trained deep learning model, wherein the one or more physiochemical properties comprises one or more of: a melting point, a boiling point, a solubility, and an Absorption, Distribution, Metabolism, Excretion, and Toxicity (ADMET) properties of the untested chemical compound.

11. The method according to claim 1, wherein the pre-trained NLP model is a transformer model.

12. The method according to claim 1, wherein the transformation of each of the plurality of SMILES notations into the plurality of non-sparse matrices via the pre-trained NLP model further comprises detecting one or more special characters present in each of the plurality of SMILES notations.

13. The method according to claim 1, wherein each of the plurality of SMILES notations is pre-processed before executing the pre-trained NLP model for the transformation of each of the plurality of SMILES notations, wherein the pre-processing of each of the plurality of SMILES notations comprises:
cleansing of each of the plurality of SMILES notations by removal of an outer layer of salt in the chemical structure;
detecting one or more outliers in each of the plurality of SMILES notations using a cluster-based local outlier factor (CBLOF);
canonicalizing each of the plurality of SMILES notations; and
augmenting the canonicalized SMILES notations.

14. The method according to claim 1, wherein the at least one chemical modification of the chemical compound comprises alteration of one or more functional groups, an atomic level variation, a change in a bond connecting an atomic pair, or any other modification in the chemical structure of the chemical compound in the drug to form the new drug.

15. A method of predicting biological activities or properties of one or more chemical compounds in a new drug, the method comprising:
receiving, by a processor, a simplified molecular-input line-entry system (SMILES) notation, wherein the SMILES notation is an indicative of a chemical structure of an untested chemical compound present in the new drug;
executing, by the processor, a pre-trained Natural Language Processing (NLP) model to transform the SMILES notation into a non-sparse matrix; and
executing, by the processor, a trained deep learning model, by passing the non-sparse matrix to the trained deep learning model to predict one or more biological activities and one or more physiochemical properties of the untested chemical compound in the new drug.

16. A system for predicting biological activities or properties of one or more chemical compounds in a new drug, the system comprising:
a processor configured to:
receive a plurality of input datasets, wherein the plurality of input datasets comprises a plurality of simplified molecular-input line-entry system (SMILES) notations associated with a plurality of drugs, wherein each of the plurality of SMILES notations is an indicative of a chemical structure of a chemical compound present in a drug of the plurality of drugs;
execute a pre-trained Natural Language Processing (NLP) model to transform the plurality of SMILES notations into a plurality of non-sparse matrices, wherein each of the plurality of non-sparse matrices is indicative of one or more biological activities of the chemical compound present in the drug,
train a deep learning model using the plurality of non-sparse matrices to obtain a trained deep learning model, wherein the trained deep learning model is used to predict one or more biological activities of an untested chemical compound in the new drug when the chemical compound in the drug is subjected to at least one chemical modification to form the new drug.

17. The system according to claim 16, wherein transformation of the plurality of SMILES notations into the plurality of non-sparse matrices comprises:
transforming each of the plurality of SMILES notations into a corresponding plurality of word embeddings by splitting each of the plurality of SMILES notations; and
transforming, via an encoder stack, the corresponding plurality of word embeddings of each of the plurality of SMILES notations into a corresponding non-sparse matrix of the plurality of non-sparse matrices, wherein the pre-trained NLP model comprises the encoder stack and a decoder stack.

18. The system according to claim 16, wherein each of the plurality of non-sparse matrices is a matrix of dimensions N×1024, wherein N is number of characters present in each of the plurality of SMILES notations.

19. The system according to claim 16, wherein the processor is further configured to pre-process each of the plurality of SMILES notations before executing the pre-trained NLP model for transformation of each of the plurality of SMILES notations, wherein the pre-processing of each of the plurality of SMILES notations comprises:
cleansing of each of the plurality of SMILES notations by removal of an outer layer of salt of the chemical structure of the chemical compound;
detecting one or more outliers in each of the plurality of SMILES notations using cluster-based local outlier factor (CBLOF);
canonicalizing each of the plurality of SMILES notations; and
augmenting the canonicalized SMILES notations.

20. The system according to claim 16, wherein the at least one chemical modification of the chemical compound comprises alteration of one or more functional groups, an atomic level variation, a change in a bond connecting an atomic pair, or any other modification in the chemical structure of the chemical compound in the drug to form the new drug.
